SEQUENCE LISTING (5 sequences)

SEQ ID NO: 1 - DNA, 1146 bp, Escherichia coli
atgatgaaaaaatcgttatgctgcgctctgctgctgacagcctctttctccacatttgctgccgcaaaaacagaacaacagattgccgatatcgttaatcgcaccatcaccccgttgatgcaggagcaggctattccgggtatggccgttgccgttatctaccagggaaaaccctattatttcacctggggtaaagccgatatcgccaataaccacccagtcacgcagcaaacgctgtttgagctaggatcggttagtaagacgtttaacggcgtgttgggcggcgatgctatcgcccgcggcgaaattaagctcagcgatccggtcacgaaatactggccagaactgacaggcaaacagtggcagggtatccgcctgctgcacttagccacctatacggcaggcggcctaccgctgcagatccccgatgacgttagggataaagccgcattactgcatttttatcaaaactggcagccgcaatggactccgggcgctaagcgactttacgctaactccagcattggtctgtttggcgcgctggcggtgaaaccctcaggaatgagttacgaagaggcaatgaccagacgcgtcctgcaaccattaaaactggcgcatacctggattacggttccgcagaacgaacaaaaagattatgcctggggctatcgcgaagggaagcccgtacacgtttctccgggacaacttgacgccgaagcctatggcgtgaaatccagcgttattgatatggcccgctgggttcaggccaacatggatgccagccacgttcaggagaaaacgctccagcagggcattgcgcttgcgcagtctcgctactggcgtattggcgatatgtaccagggattaggctgggagatgctgaactggccgctgaaagctgattcgatcatcaacggcagcgacagcaaagtggcattggcagcgcttcccgccgttgaggtaaacccgcccgcccccgcagtgaaagcctcatgggtgcataaaacgggctccactggtggatttggcagctacgtagccttcgttccagaaaaaaaccttggcatcgtgatgctggcaaacaaaagctatcctaaccctgtccgtgtcgaggcggcctggcgcattcttgaaaagctgcaataa

SEQ ID NO: 2 - DNA, 25 bp, Artificial
  Synthetic primer Citro strt F
atgatgaaaaaatcgatatgctgcg

SEQ ID NO: 3 - DNA, 25 bp, Artificial
  Synthetic primer Citro_strt_R
caaacagaccaatgctggagttagc

SEQ ID NO: 4 - DNA, 23 bp, Artificial
  Synthetic primer Citro_F
tgctgctgacagcctctttctcc

SEQ ID NO: 5 - DNA, 20 bp, Artificial
  Synthetic primer Citro_R
ggcgggtttacctcaacggc

(19) United States
(12) Patent Application Publication
(10) Pub. No.: US 2013/0029863 A1
(43) Pub. Date: Jan. 31, 2013

(54) METHOD FOR DETECTING MICROORGANISMS

(71) Applicants: Etienne Ruppe, Paris (FR); Antoine Andremont, Malakoff (FR); Raymond Ruimy, Montigny Les Cormeilles (FR); Patrice Courvalin, Paris (FR); Sylvie Bremont, Noisy Le Sec (FR)

(72) Inventors: Etienne Ruppe, Paris (FR); Antoine Andremont, Malakoff (FR); Raymond Ruimy, Montigny Les Cormeilles (FR); Patrice Courvalin, Paris (FR); Sylvie Bremont, Noisy Le Sec (FR)

(73) Assignees: Institut Pasteur, Paris (FR); Assistance Publique - Hopitaux de Paris, Paris (FR)

(21) Appl. No.: 13/499,533

(22) PCT Filed: Sep. 30, 2010
(86) PCT No.: PCT/EP2010/064589

§ 371 Date: Oct. 9, 2012

(30) Foreign Application Priority Data
Sep. 30, 2009 (FR) 09/04677

(51) Int. Cl. C12Q 1/68; C40B 40/06; C40B 30/04

(52) U.S. Cl. 506/9; 435/6.15; 435/6.12; 435/6.11; 506/16

(57) ABSTRACT

The invention relates to the diagnostic field and to the characterization of antibiotic-resistant bacteria in a sample by detecting specific nucleotides in the CMY-2 gene.

The invention relates to the diagnostic field and to the detection of antibiotic-resistant bacteria in a sample.

The activity of antibiotics tends to become reduced over time owing to the appearance and spread of bacterial resistance mechanisms. Today, introduction on the market of new effective antibacterial molecules is greatly slowed, while the extensive use of antibiotics in human and veterinary medicine has led to the massive and globalized spread of the determinants of bacterial resistance.

β-lactam antibiotics constitute the family of antibiotics most widely consumed throughout the world owing to their broad spectrum of antibacterial activity, their efficacy and the fact that they are virtually free of adverse effects. Consequently, the mechanisms of resistance to these antibiotics are widespread in the bacterial world, in particular among pathogenic bacteria, namely enterobacteria.

The most widespread mechanism of resistance to β-lactam antibiotics among them is the production of hydrolyzing enzymes called β-lactamases. These enzymes constitute a family that is very heterogeneous in terms of spectrum of hydrolysis and of nucleotide sequence. The genes encoding these enzymes are located on the chromosome of bacteria or on mobile elements such as plasmids. This carrier that can be transmitted from bacterium to bacterium means that there is considerable spread of the resistance gene. While the origin of most β-lactamases remains unknown at this time, it has recently been possible to observe the mobilization of genes of chromosomal origin on mobile elements, including plasmid cephalosporinases.

Cephalosporinases have a broad spectrum of hydrolysis, and the treatment of infections caused by bacteria producing such enzymes is restricted to a few molecules (carbapenems), the activity of which can be compromised if other mechanisms of resistance exist.

The spread of cephalosporinase genes in pathogenic bacteria such as Escherichia coli is a major public health problem, in the same way as the spread of extended-spectrum β-lactamases (ESBLs). The only effective means for combating this spread within health facilities is to detect the patients carrying bacteria producing such enzymes and to take the necessary hygiene and confinement steps to limit patient-to-patient transmissions. The detection of multiresistant bacteria in the nasal flora (methicillin-resistant Staphylococcus aureus, or MRSA) or the digestive flora (ESBL-producing enterobacteria, vancomycin-resistant enterococci, or VRE) is today routine practice in many health facilities. However, most of the bacteria which produce plasmid cephalosporinases are not recognized by these methods, allowing the unseen spread of these genes.

Many authors have proposed various phenotypic methods for demonstrating the production of cephalosporinases in bacterial strains (1, 3-11). However, the chromosomal or plasmid position of the resistance gene (essential in terms of spread impact and therefore in terms of hygiene steps to be implemented) can be determined only by molecular methods for bacteria capable of producing their own chromosomal cephalosporinase. Indeed, the nucleotide sequences are virtually identical between the plasmid-positioned genes and the chromosomal genes. Consequently, if the “parental” bacterium is frequently present in the flora of patients, the risk is that this will result in a large number of false positives with regard to the detection of plasmid cephalosporinases.

The present invention provides a method for detecting the presence, in a sample, of a bacterium carrying a plasmid cephalosporinase gene of CMY-2 type, in the presence or absence of the “parenteral” bacterium Citrobacter freundii.

This detection is now possible through the demonstration, by the inventors, that the CMY-2 gene of plasmid origin has a cytosine (respectively a guanine) in position 373 (respectively 437), the gene of chromosomal origin having an adenosine (respectively a cytosine).

The invention thus relates to a method for detecting the presence, in a sample, of at least one bacterium which is resistant to β-lactam antibiotics and which carries a plasmid CMY-2 gene, characterized in that it comprises the step of detecting the presence of a DNA molecule in said sample, the sequence of which exhibits at least 90% identity, preferably at least 95%, more preferably at least 98%, most preferably at least 99% identity with SEQ ID No. 1, and nucleotides 373 and 437 of which are respectively a cytosine and a guanine.

SEQ ID No. 1 thus represents a plasmid allele of the CMY-2 gene. Its sequence can vary owing to genetic diversity. SEQ ID No. 1 corresponds to GenBank accession AM779745, corresponding to the blaCMY-2 gene for CMY-2 β-lactamase, isolated from the Escherichia coli pta plasmid.

Other plasmid CMY-2 genes are also available in the databases, under various accession numbers.

The presence of these two nucleotides (C373 and G437) in the CMY-2 gene is indicative of the plasmid nature of the gene. Indeed, the genes of chromosomal origin have, respectively, an adenosine and a cytosine.

The consequence of these mutations is the presence of an arginine in position 125 or 146 in the proteins expressed by the plasmid gene, the genes of chromosomal origin expressing a protein comprising a serine in position 125 and a threonine in position 146.

It should be noted that the method according to the invention theoretically makes it possible to detect the presence of a single bacterium containing a plasmid CMY-2 in the sample (with satisfactory amplification conditions).

However, the method according to the invention does not make it possible to determine whether the bacteria carrying the plasmid CMY-2 gene are all of the same type or whether there are various types of bacteria containing the plasmid in the sample. Supplementary tests are necessary in order to specify this point and to characterize the nature of the bacterium carrying the resistance.

The sample may be of any type. It may be a biological sample which has previously been taken from a patient. Such a sample may be, without this list being exhaustive, a saliva sample, a urine, fecal or blood sample, or a sample resulting from a biopsy, in particular from an intestinal biopsy.

The method may also be carried out on a specimen taken from a food sample, from a surface or from an implement, in particular one that can be used in the hospital environment (verification of the asepsis of the surface or of the implement).

The method according to the invention thus makes it possible to determine the presence of bacteria containing a CMY-2 gene of plasmid origin, including when the sample contains bacteria of the genus Citrobacter freundii.

Indeed, it is in fact probable that the plasmid CMY-2 gene is derived from the chromosomal AmpC gene of C. freundii (2). This AmpC gene has a sequence close to SEQ ID No. 1 (identity greater than 80% with SEQ ID No. 1, and which can be greater than 90% for certain alleles), but nucleotides 373 and 437 of which are respectively an adenosine and a cytosine. It should be noted that the variability which exists between the various chromosomal genes is greater than that which exists between the various plasmid genes.

The percentage identity between two nucleotide sequences is determined by comparing the two sequences optimally aligned, on a comparison window.

In the context of the present invention, the two sequences are thus aligned over all of the 1143 nucleotides corresponding to the sequence encoding the protein for resistance to β-lactam antibiotics (1146 counting the final stop codon). The percentage identity is thus calculated by the following formula: percentage identity=((number of differences between the two sequences)/1143)×100.

Known algorithms can enable the differences between two sequences to be detected in an automated manner, and thus allow the percentage identity to be measured.

In one preferred embodiment, said polymorphism detection step comprises a step of amplification of the DNA present in said sample. This in fact makes it possible to detect the presence of a very low number of bacteria.

This amplification is carried out by polymerase chain reaction, PCR, using any primer that may be designed by those skilled in the art.

Use is preferably made of the primers Citro strt F (5′-ATGATGAAAAAATCGATATGCTGCG-3′, positions 1-25, SEQ ID No. 2) and Citro_strt_R (5′-CAAACAGACCAATGCTGGAGTTAGC-3′, positions 511-535, SEQ ID No. 3) amplifying a 535 bp fragment.

Use may also be made of the primers Citro_F (5′-TGCTGCTGACAGCCTCTTTCTCC-3′, positions 29-51, SEQ ID No. 4) and Citro_R (5′-GGCGGGTTTACCTCAACGGC-3′, positions 952-971, SEQ ID No. 5), amplifying a 942 bp fragment.

Use may also be made of the pair of primers SEQ ID No. 2—SEQ ID No. 5.

Those skilled in the art are able to design other types of primers for amplifying the CMY-2 gene. However, the inventors have shown that the above primers exhibit good efficiency.

It is possible to carry out an amplification with a single pair of primers, or to carry out two series of PCR, the second amplification being carried out with primers located inside the first fragment amplified.

A high-affinity or ultra-high affinity DNA polymerase is preferably used. This is because, since the specific presence of defined nucleotides is being sought, it is important for the polymerase not to generate mutations in the amplified fragment. Such enzymes are commercially available. Mention may thus be made of the Phusion™ DNA polymerase enzymes (Finnzymes Oy, Espoo, Finland) which have an error rate of about 4.4×10⁻⁷.

When the amplification reaction is carried out, it may be chosen to amplify only the CMY-2 gene, using an appropriate pair of primers, or to amplify, in the same reaction, various genes potentially present in the sample (in particular other genes for resistance to various antibiotics), using a plurality of different primers. Several pairs of primers, each being specific for a gene that it is desired to amplify, are thus introduced into the reaction tube.

The detection of the polymorphic nucleotides, indicating the presence of the plasmid CMY-2 gene, is carried out by any manner known in the art. Those skilled in the art are in fact aware of many suitable protocols which can be identified on Internet search engines. Mention may in particular be made of www.ipbs.fr/formation/biotech/mutations.pdf.

The DNA fragment can thus be sequenced, in particular by the Sanger sequencing method using only three deoxynucleotides and one dideoxynucleotide and one primer (in particular a primer as mentioned above). The sequencing products are then separated on a polyacrylamide gel and the presence of the mutation is confirmed by observing the size of the products.

A primer extension reaction can also be carried out. In this case, the primer used is defined such that the first nucleotide to be incorporated after this primer corresponds to the polymorphic nucleotide.

However, preferably, said detection step comprises a step of hybridization of the DNA (preferentially amplified) with a probe specific for either mutation, or for both mutations. The hybridization of two DNA chains is due to the pairing of complementary bases A-T and G-C and is reduced when there is mismatching. The Tm of an oligonucleotide of from 10 to 20 bases will therefore be decreased if there is a mismatch. In order to calculate the Tm of an oligonucleotide of less than 30 nucleotides, the following relationship is used: Tm=2(A+T)+4(G+C), in which A, T, G and C are respectively the number of each of these bases in the oligonucleotide.

The “dot blot” method can thus be used, by attaching a probe oligonucleotide carrying the mutation to a nitrocellulose membrane, and applying the DNA present in the sample (or the amplified DNA) under conditions which allow the specific hybridization of the polymorphism (the Tm is calculated according to the composition of the probe oligonucleotide selected).

In this case, the probe is attached to a solid support.

In one particular embodiment, the hybridization is carried out on a DNA chip. A first spot contains an oligonucleotide which has the polymorphism of the chromosomal AmpC gene of Citrobacter freundii, and a second spot contains an oligonucleotide which has the polymorphism of the plasmid CMY-2 gene. The DNA is amplified and labeled with a fluorophore. It is then hybridized on the oligonucleotides attached to the chip. A fluorescence is observed if there is hybridization.

It is also possible to detect the presence of the two nucleotides mentioned above by other methods. After amplification, the amplification product can be mixed with DNA which does not comprise the mutations and these two oligonucleotides can be hybridized. The mismatches are then detected by enzymatic cleavage (in particular using Cel I) or chemical cleavage. This detection method is well known and suitable enzymes are in particular described in EP 964929.

The invention also relates to a kit for detecting the presence of at least one bacterium which is resistant to β-lactam antibiotics in a sample, said bacterium having a plasmid CMY-2 gene.

The kit according to the invention may comprise primers for amplifying the CMY-2 gene. It may also comprise probes for identifying the presence of a cytosine in position 373 and/or a guanine in position 437, the positions being determined relative to SEQ ID No. 1. The probes may be present in dehydrated form. Alternatively, the kit may contain a DNA chip comprising said probes. The kit may also contain enzymes for amplifying the target DNA, and elements for labeling the DNA. Preferably, the kit according to the invention also contains instructions for carrying out the method as described above.

### EXAMPLES

### Example 1

The nature of nucleotides 373 and 437 of the chromosomal ampC gene of 83 strains of C. freundii of varied origins was investigated: 62 clinical strains from the Bichat-Claude Bernard hospital (Assistance Publique des Hôpitaux de Paris [state-owned Paris hospitals]) isolated between October 2006 and January 2009, 18 strains from the Antimicrobial Agent Unit of the Pasteur Institute of Paris, and 4 strains isolated from the digestive flora of Wayampi Indians living in French Guyana (“ERAES” project, AFFSET [French Agency for Environment Security]).

These strains were identified as C. freundii by conventional methods.

The nucleotides of the ampC genes present in the databases (GenBank accession numbers): CFRBL, CFRBLAC, X03866, X51632, Y15129, X76636, AF349569, AF349570, AF746169, AF125469 and AF426097, were also studied.

The ampC gene of C. freundii has 1143 base pairs (bp) (without taking into account the STOP codon).

The analysis of these genes shows that nucleotide 373 of each of these genes is an adenosine. Nucleotide 437 of each of these genes is a cytosine.

Conversely, all the genes having a cytosine at position 373 and a guanine at position 437 are of plasmid origin: see, in particular, the following genes (GenBank assession numbers): AM779745, AM779746, AM779747, AM779748, AY899923, AY899924, AY899925, AY899926, AY899927, AY899928, AY899929, DQ173299, DQ355981, DQ478718, EF406116, EU113220, EU113221 and EU113222.

These results show that the presence of the nucleotides C373 and G437 in a CMY-2 gene indicates that it is of plasmid origin.

1. A method for detecting the presence, in a sample, of a bacterium which is resistant to β-lactam antibiotics and which carries a plasmid CMY-2 gene, characterized in that it comprises the step of detecting the presence, in said sample, of a DNA molecule, the sequence of which exhibits at least 90% identity with SEQ ID No. 1, and nucleotides 373 and 437 of which are respectively a cytosine and a guanine.

2. The method as claimed in claim 1, characterized in that said sample contains bacteria of the genus Citrobacter freundii.

3. The method as claimed in claim 1, characterized in that said detection step comprises a step of amplification of the DNA present in said sample.

4. The method as claimed in claim 1, characterized in that said detection step comprises a step of hybridization of the DNA with a probe specific for either mutation.

5. The method as claimed in claim 4, characterized in that said probe is attached to a solid support.

6. A kit for detecting the presence, in a sample, of a bacterium which is resistant to β-lactam antibiotics and which carries a plasmid CMY-2 gene, comprising instructions for carrying out a method as claimed in claim 1, and also primers for amplifying the CMY-2 gene or a probe for identifying the presence of a cytosine in position 373 and/or a guanine in position 437, the positions being determined relative to SEQ ID No. 1.
